(19) United States
(12) Patent Application Publication
(10) Pub. No.: US 2010/0115548 A1
(43) Pub. Date: May 6, 2010

(54) PATIENT ENTERTAINMENT SYSTEM WITH SUPPLEMENTAL PATIENT-SPECIFIC MEDICAL CONTENT

(75) Inventor: Evgeniy Leyvi, Riverdale, NY (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/525,914

(22) PCT Filed: Jan. 25, 2008
(86) PCT No.: PCT/IB2008/050280

§ 371 Date: Aug. 5, 2009

Related U.S. Application Data
(60) Provisional application No. 60/888,772, filed on Feb. 8, 2007.

(51) Int. Cl. H04N 7/025; A61B 5/00

(52) U.S. Cl. 725/34; 600/300

(57) ABSTRACT

A medical information delivery system configured for operation in conjunction with an entertainment device (30) configured to present an entertainment audio/video content datastream (26) includes a multiplexor (56) for inserting additional content into the entertainment audio/video content datastream, and a controller (42) for selecting patient-specific content for insertion into the entertainment audio/video content datastream. A medical information delivery method includes presenting an entertainment audio/video content datastream (26) via an entertainment device (30), and presenting patient-specific content via the entertainment device with the presenting of the entertainment audio/video content datastream.

The following relates to the medical arts. It especially relates to providing medical advice to patients in a hospital or other clinical setting, and is described with illustrative reference thereto. The following relates more generally to providing medical advice to patients or other subjects in hospital, home, or other settings.

It is known to provide monitoring of a patient in a hospital. Such monitoring may include automated measurement of physiological parameters such as heart rate, blood pressure, or SpO₂ level, or less automated tracking of patient weight, food intake, bowel movements, urinary function, exercise, sleep periods, or so forth. Such information is typically recorded by hospital personnel automatically (for example, by storing trending information relating to measured physiological parameters) or by recording such information in electronic or paper-based patient charts. This information is used by physicians, nurses, or other care providers to tailor the patient's care to the current physical, and perhaps mental, condition of the patient.

It is also recognized that this information is advantageously conveyed to the patient, so as to enable the patient to alter his or her behavior accordingly. For example, a nurse may tell a patient that he or she needs more sleep, or more exercise, or should eat more or less, or so forth. The patient monitoring in raw form (such as heart rate data, food intake charting, or so forth) is typically of little value to the patient. Rather, a doctor, nurse, or other care provider typically filters the raw patient monitoring data by suitable analysis to construct appropriate behavioral modification recommendations. This filtering can be complex (e.g., analysis of an electrocardiogram to determine cardiac condition) or straightforward (e.g., comparing food intake with a caloric intake target).

Unfortunately, the conveyance of information to the patient is sometimes ineffective or inefficient. A typical patient may receive brief nurse visits a few times a day, and perhaps a brief visit from a physician. During these brief patient-caretaker encounters, the caretakers attempt to convey relevant information or advice to the patient. However, the nurse or physician typically has one or more specific tasks to perform during the brief visit, such as changing an intervenous drip, performing diagnostic probing, or so forth, and may therefore be distracted and fail to provide the patient with relevant information or advice.

Still further, even if the patient receives the information or advice, he or she may forget or ignore the information or advice. Repetitive communication would enhance the patient's memory and likely increase the chance of patient follow-through. However, staffing limitations and other tasks impose limitations on the amount of patient-caretaker interaction time.

The following provide improvements addressing the above-referenced problems and others.

In accordance with one aspect, a medical information delivery system is configured for operation in conjunction with an entertainment device configured to present an entertainment audio/video content datastream. The medical information delivery system includes means for inserting additional content into the entertainment audio/video content datastream, and means for selecting patient-specific content for insertion into the entertainment audio/video content datastream.

In accordance with another aspect, a medical information delivery method is disclosed, including presenting entertainment audio/video content via an entertainment device, and selectively presenting patient-specific content via the entertainment device with the presenting of the entertainment audio/video content.

In accordance with another aspect, an information delivery system is disclosed, including an entertainment device comprising one or more components configured to present an entertainment audio/video content datastream, and one or more additional components configured to select and insert viewer-specific content unrelated to the entertainment content into the entertainment audio/video content datastream for presentation by the entertainment device along with the entertainment audio/video content datastream.

One advantage resides in presenting patient-specific information or advice in conjunction with audio/video content presentation of interest to the patient.

Another advantage resides in leveraging existing audio/video equipment to provide patient-specific information and advice in an automated manner.

Another advantage resides in leveraging available entertainment-oriented audio/video equipment to additionally provide patient-specific information and advice.

Another advantage resides in providing medical information or advice to a patient using a television or entertainment system with which the patient is familiar and comfortable.

Another advantage resides in providing comprehensible medically-related advice to a patient based on real-time patient monitoring.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

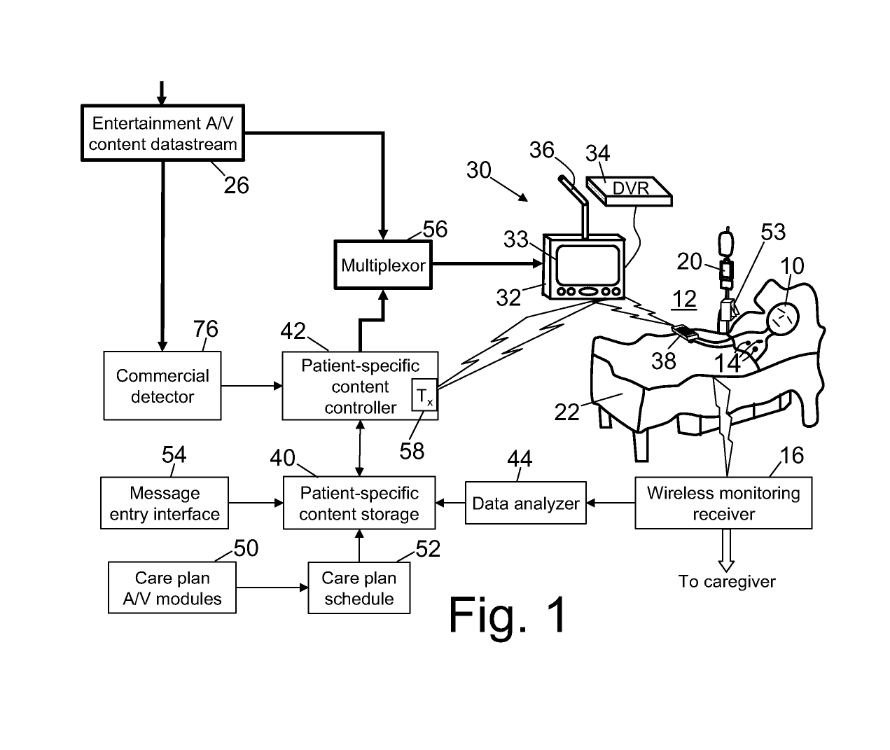

FIG. 1 diagrammatically depicts a patient in a hospital room, an entertainment device in the hospital room including a television and a digital video recorder, and a medical information delivery system configured for operation in conjunction with the entertainment device.

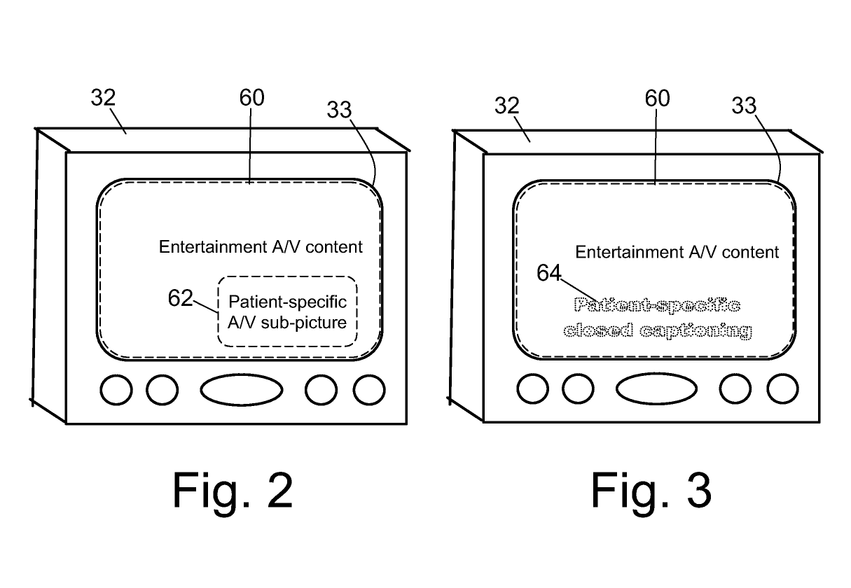

FIGS. 2-8 diagrammatically depict various illustrative ways the medical information delivery system of FIG. 1 can insert patient-specific content into an entertainment audio/video content datastream being presented by the entertainment device.

With reference to FIG. 1, a patient 10 is disposed in a hospital room 12 or other clinical or home setting. The patient 10 is optionally monitored, for example using a body sensor network comprised of wireless sensors 14. The sensors 14 may monitor, for example, cardiac cycling or heart rate, blood pressure, SpO₂, respiration or respiratory rate, or so forth. The wireless sensors 14 communicate wirelessly with a wireless monitoring receiver 16 that collects patient measurements. The wireless communication employs a suitable wireless protocol such as Bluetooth, Zigbee, WiFi, or so forth. To facilitate patient mobility, for example to enable the patient to go outdoors, the sensors may be integrated into the patient's clothing or otherwise attached to the patient in a way that promotes patient mobility. The collected patient measurements may be forwarded to a caregiver, for example by transmission to a nurses' station that collects and stores patient measurements. In some embodiments, the wireless sensors 14 also intercommunicate wirelessly amongst each other to form a body sensor network that optionally operates autonomously even if the patient 10 (and hence the sensors 14) move away from and out of range of the wireless monitoring receiver 16. Although wireless sensors 14 are illustrated, in other embodiments wired sensors, or a combination of wired and wireless sensors, may be used.

The patient 10 additionally or alternatively optionally receives therapy or treatment, such as an illustrated intravenous (IV) fluid drip 20. In a hospital setting, the patient 10 typically spends a significant amount of time in a bed 22. When not sleeping, the patient will typically watch an entertainment audio/video content datastream 26 displayed by an entertainment device 30, such as a television 32 having a display or screen 33 for displaying video, and optionally including additional components such as an illustrated digital video recorder 34. The illustrated television 32 is ceiling-mounted by an articulated mounting rod 36; alternatively, the television 32 may be disposed on a stand, floor-mounted, or otherwise supported. Typically, the patient 10 can operate the television 32, digital video recorder 34, or other components of the entertainment device 30 using at least one hand-held remote controller 38.

In some embodiments, the television 32 is a commercially available cathode ray tube television, LCD television, plasma television, front- or rear-projection television, or so forth. The television 32 can utilize substantially any audio technology, such as one or more built-in speakers, a wired or wireless sound system including two or more external speakers, incorporation of a stereo or high fidelity audio system, or so forth. The entertainment audio/video content datastream 26 can take various forms, including for example: a wireless broadcast signal; a cable television feed carrying television broadcasts; a satellite television feed carrying television broadcasts; a closed-circuit television broadcast that services residents of an assisted care community or patients of a hospital; or a non-broadcast audio/video content datastream such as a video cassette recorder output; a DVD output; an output from a digital video recorder; various combinations or juxtapositions of these; or so forth. The entertainment audio/video content datastream can be standard definition (e.g., standard definition television, typically denoted as SDTV), high definition (e.g., high definition television, typically denoted as HDTV), or another resolution, and can employ a standard 4:3 display, widescreen display, or other display size. Moreover, in some embodiments the entertainment device does not include a television. For example, the entertainment device can be a portable DVD player that plays back DVDs, in which case the entertainment audio/video content datastream includes the DVD output. The display can also be a computer or gaming monitor, a personal data assistant (PDA), a cellular telephone, or so forth. At least one component of the entertainment device 30 should include a display, such as the illustrated display or screen 33 of the illustrated television 32, or the display of a PDA or cellphone, or so forth.

As used herein, the term “audio/video content” or the like is intended to encompass both video-only content and combined audio and video content. For example, “audio/video content” may include displayed text, silent video, a displayed static image, video with an accompanying soundtrack, or so forth.

In the illustrated embodiment, the entertainment device 30 is leveraged to additionally provide the patient 10 with patient-specific content delivered with the entertainment audio/video content datastream 26. Advantageously, the patient 10 typically is interested in the entertainment audio/video content datastream 26, and hence is watching and listening to the entertainment device 30. Thus, when this entertainment device 30 delivers patient-specific content such as medical information or advice, the patient 10 is likely to perceive this information and advice. Moreover, in some embodiments the patient 10 is largely confined to (or at least spends a lot of time in) the hospital room 12 or another limited setting such as an assisted living community, a house in which the patient is house-bound, or so forth, and the patient likely spends a substantial amount of time watching the entertainment audio/video content datastream 26 via the entertainment device 30. Thus, the patient-specific content such as behavior modification advice can be delivered continuously or on a periodic or other frequent basis—as a result, the patient 10 perceives this information or advice frequently and is likely to remember it and, ideally, act upon it. The behavior modification advice can be repeated to allow the patient to access it when desired, or to provide repetitive reinforcement to enhance the patient's learning of the content. Still further, in some embodiments the delivered patient-specific information is constructed in real time based on patient medical readings provided by the sensors 14, which enables the provided information or advice to be timely.

With continuing reference to FIG. 1, a patient-specific content storage 40 stores patient-specific content that is selectable by a patient-specific content controller 42. The patient-specific content can be generated or supplied in various ways. In one approach, a data analyzer 44 analyzes patient measurements acquired by the sensors 14 and wireless monitoring receiver 16, or acquired by another wired or wireless sensor. The data analyzer 44 analyzes the patient measurements to generate information or advice in comprehensible language that is readily understood by the patient 10 even if the patient 10 has little or no specialized medical knowledge or expertise. For example, the data analyzer 44 may analyze heart rate measurements, blood pressure measurements, or other physiological measurements and determine whether the patient is in an excited state. If the patient is excited, and has a heart condition or other condition that makes the patient susceptible to a heart attack or other stress-aggravated trauma, then the data analyzer 44 generates patient-specific content to alleviate the stress. This patient-specific content may include textual advice such as “Calm down, you're getting too excited”, or a soothing static visual image such as a cloudscape, or a soothing video image such as an image of fish swimming in an aquarium, or a suggestion to perform a calming ritual, so forth. If the data analyzer 44 determines that the patient is undergoing an asthma attack, it may generate a patient-specific message such as “Please use your rescue inhaler immediately”. As another example, the data analyzer 44 may receive a patient food intake record from a hospital network or elsewhere. The data analyzer 44 compares this food intake record with a desired food intake profile (e.g., desired caloric intake, desired fruit/vegetable-to-meat ratio, or so forth) and constructs recommendations for dietary modifications that serve as patient-specific content. In some embodiments, the data analyzer 44 may provide the patient with feedback on recommended choices for meals. For example, the data analyzer 44 may recommend that a patient who has been eating too little fruit should elect the “apple sauce” side item from the lunch menu, rather than the “french fries” side item.

As another example, the data analyzer 44 may receive a weight measurement, and compare this weight measurement with a target weight for the patient 10. If, for example, the weight measurement is too low, the data analyzer 44 constructs patient-specific advice content such as “You are still not gaining enough weight after your illness. You need to eat more high calorie foods.” The weight measurement can be acquired by directly reading a suitable sensor, such as a scale, when the patient is being weighed, or by reading a weight measurement that is manually input, for example entered by a nurse into an electronic patient chart that is accessible to the data analyzer 44.

Another contemplated source of patient-specific content are pre-recorded audio/video content that may be arranged, for example, as one or more care modules 50. Each care module 50 includes audio/video content directed toward a particular medical issue, and the physician or other care giver selects one or more of the care modules 50 that are appropriate for the condition of the patient 10 as patient-specific content to be presented to the patient 10 in accordance with a care plan schedule 52. For example, if the patient 10 has recently suffered a heart attack, then the care plan schedule 52 may include a weight loss care plan module, a “quit smoking” care plan module, and a “get started with walking” care plan module as patient-specific content. As used herein, a given care plan module selected for presentation to the patient 10 is considered to be patient-specific content since it is selected for the patient 10, although it is to be recognized that the same care plan module might also be independently selected for presentation to another patient.

Another contemplated source of patient-specific content is a message entry interface 54, via which a physician, nurse, or other caregiver can input patient-specific content intended for delivery to the patient 10 via the entertainment device 30. For example, the doctor may enter a message such as “Don't forget to take at least three walks each day down the hospital hallway” so as to encourage a post-surgery patient 10 to being to move about after surgery. As another example, reminders to take medications can be displayed at appropriate times.

The patient-specific content can also relate to information informing the patient about an action he or she should take. For example, an automatic pills dispenser 53 can be mounted on the IV stand or in another position within convenient reach of the patient. The patient specific-content may in this example be a message telling the patient that a scheduled administration of medicine in a pill form is available from the dispenser 53. Such scheduling is suitably incorporated into the care plan schedule 52. When the appointed time arrives for a pill dispensation, the patient-specific content controller 42 or another device outputs a wired or wireless triggering signal to the automatic pill dispenser 53, which outputs a pill using a vending machine-type mechanism. Optionally, the automatic pill dispenser also provides an audibly and/or visually perceptible indication of the availability of the dispensed pill, for example as a light, buzzer sound, or so forth. Concurrently presented patient-specific content includes an audio/video message displayed on the television 32 notifying the patient of the scheduled medication intake and instructing the patient on where the pill is and how to take it (for example, with food, or with a full glass of water, or so forth).

The patient-specific content controller 42 selects content from the patient-specific content storage 40 for presentation to the patient with the entertainment audio/video content datastream 26. The controller 42 selects what content to present, when to present it, and optionally how to present it. The patient-specific content storage 40 may serve as long-term storage for storing patient-specific content for hours, days, or even longer time intervals before delivery. In other embodiments, the patient-specific content storage 40 may be a short-term buffer that stores the patient-specific content for a short time interval sufficient for the controller to arrange for its presentation. In other embodiments, the patient-specific content storage 40 serves as both long-term storage for less urgent data and short-term buffering for urgent data that should be presented as soon as possible (such as, for example, the message “Please use your rescue inhaler immediately” displayed in response to an asthma attack).

The patient-specific content controller 42 selects content from the patient-specific content storage 40 for presentation. A multiplexor 56 inserts the selected patient-specific content into the entertainment audio/video content datastream 26 such that it is presented by the presentation device 30. The content controller 42 and multiplexor 56 can operate in various ways to insert the patient-specific content into the entertainment audio/video content datastream 26. The selected content insertion method depends upon the manner in which the patient-specific content is to be presented, and may further depend on the capabilities of the television 32 or other component or components of the audio/video entertainment device 30. Some contemplated content insertion approaches take advantage of the wireless remote control capability incorporated into most audio/video entertainment system components. For example, the illustrated television 32, like most televisions, is controllable by the hand-held remote controller 38 that communicates with the television via an infrared, radio frequency, or other wireless signal. In the illustrated embodiment, the hand-held remote controller 38 is a “universal” remote, that also controls the DVR 34. In other embodiments, separate handheld remote controllers may be provided for different components of the entertainment device, or some components may not be remotely operable. The hand-held remote controller 38 has the capability, by transmitting appropriate wireless signals selected by buttons or other user operable inputs of the remote controller 38, to turn the television 32 on or off, to select the channel, to adjust the audio volume, to turn subtitling on or off, and so forth. The content controller 42 can therefore control the television or other component of the audio/video entertainment device 30 by operating a wireless transmitter 58 to mimic these wireless signals to cause the television or other component of the audio/video entertainment device 30 to turn on or off, to switch to a desired channel, to turn subtitling on or off, or so forth. Typically, the signal from the wireless transmitter 58 takes priority over any signal produced by the handheld remote controller 38. Some illustrative examples of methods for inserting patient-specific content into the entertainment audio/video content datastream 26 are described. Analogously, the patient can communicate with a transmitter/receiver via the controller 38 to confirm that a walk or medications were taken, a length of the walk, or so forth.

With continuing reference to FIG. 1 and with further reference to FIG. 2, in one approach the entertainment audio/video content datastream 26 is displayed in a main window 60 (indicated by a dashed-line border in the FIGURES) that occupies most of the space of the video display screen 33, while the patient-specific audio/video content is displayed in a superimposed patient-specific audio/video sub-window 62 (indicated by a dashed-line border in FIG. 2). One approach for implementing this type of content insertion makes use of the built-in picture-in-picture capability of some televisions. If the television 32 has picture-in-picture capability, then insertion of the patient-specific content into a sub-window can be achieved as follows. A channel is designated for the patient-specific content, and the multiplexor 56 loads the patient-specific content by modulating the content into the frequency band of the designated channel Format conversion, interpolation to change pixel resolution, or other format adjustment is performed as appropriate. The controller 42 operates the transmitter 58 to send suitable mimicked remote control signals to configure the television 32 to display the sub-window 62 (that is, a “picture within a picture”) with the sub-window 62 tuned to the designated channel carrying the patient-specific content. The patient 10 optionally can use the hand-held remote controller 38 to manipulate the sub-window 62 in accordance with the built-in picture-in-picture capabilities of the television 32. For example, with some televisions, the patient 10 may be able to move the sub-window 62 to a selected corner of the screen 33, or may be able to re-size the sub-window 62, or may be able to turn switch the sound output between the audio portion of the entertainment datastream 26 and an optional audio portion of the patient-specific content, or so forth. If allowing the patient to manipulate (and perhaps turn off) the sub-window 62 is undesirable, then the controller 42 can be configured to have the transmitter 58 re-send the mimicked remote control signals configuring the television 32 to display the sub-window 62 tuned to the designated channel carrying the patient-specific content on a recurring basis, such as every ten seconds or so.

With continuing reference to FIG. 1 and with further reference to FIG. 3, in another approach the patient-specific content is displayed as subtitling 64 that is superimposed on the main window 60 that occupies most of the space of the display or screen 33. Again, the main window 60 displays the video content of the entertainment audio/video content datastream 26. One approach for implementing this type of content insertion makes use of the built-in closed captioning capability provided in some televisions to allow textual display of dialog for the hearing impaired. If the television 32 has built-in closed captioning capability, then insertion of the patient-specific content as closed captioning can be done as follows. The multiplexor 56 inserts the patient-specific content into the entertainment audio-video content datastream 26 as a closed captioning data, in the format ordinarily used for inserting closed captioning information. The controller 42 then operates the transmitter 58 to send suitable mimicked remote control signals to configure the television 32 to enable the built-in closed captioning display. In some embodiments, the patient 10 can elect to turn off the closed captioning using the hand-held remote controller 38. If allowing the patient 10 to turn off the patient-specific closed captioning content 64 is undesirable, then the controller 42 can operate the transmitter 58 to re-send the mimicked remote control signals configuring the television 32 to display closed captioning on a recurring basis, such as every ten seconds or so.

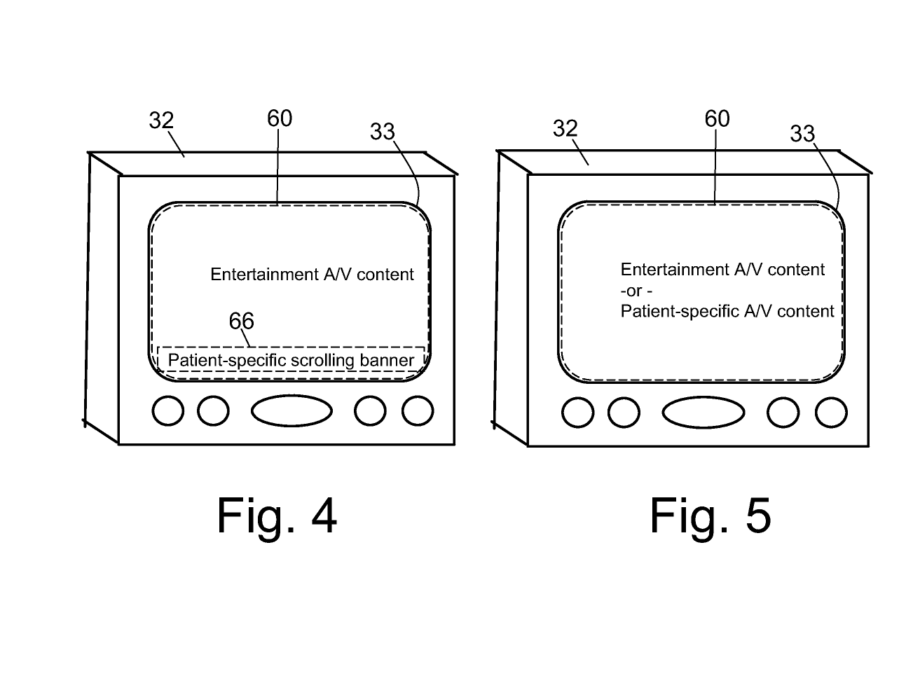

With continuing reference to FIG. 1 and with further reference to FIG. 4, in another approach the patient-specific content is displayed as a horizontally scrolling banner 66 that is superimposed on the main window 60 that occupies most of the display or screen 33. Again, the main window 60 displays the video portion of the entertainment audio/video content datastream 26. This type of content insertion is suitably performed at the datastream level. The video portion of the entertainment datastream 26 is processed to substitute pixels corresponding to the scrolling banner 66 for pixels of the entertainment datastream 26 in the region of the scrolling banner 66. The modified content is then displayed in the main window 60, with the scrolling banner 66 appropriately superimposed by the pixel-level video data substitution. If the entertainment audio/video content datastream 26 includes multiple channels (such as in some cable or satellite television feeds) then the pixel-level data substitution may be performed on each channel, or a feedback path (not shown) may be provided to tell the multiplexor 56 which channel is currently being viewed, so that the substitution is made only in the video portion of the currently viewed channel.

With continuing reference to FIG. 1 and with further reference to

FIG. 5, in another approach a time-domain multiplexing is used. That is, in these approaches the main window 60 presents either the entertainment audio/video content datastream 26 or the patient-specific substitute datastream, and the controller 42 switches between the entertainment and patient-specific content datastreams. One approach for implementing this type of content insertion is to designate a channel for the patient-specific content. The multiplexor 56 loads the patient-specific content by modulating the content into the frequency band of the designated channel Format conversion, interpolation to change pixel resolution, or other format adjustment is performed as appropriate. The controller 42 operates the transmitter 58 to send suitable mimicked remote control signals to switch to the designated channel when the controller 42 decides that patient-specific content should be presented. When the controller 42 decides that the patient-specific content should be turned off, it suitably operates the transmitter 58 to send a remote control signal mimicking the hand-held remote controller's “switch to last channel” operation to return to the entertainment content datastream 26. This “switch to last channel” operation is provided in most commercially available television hand-held remote controllers to switch to the last displayed channel For example, if the patient 10 is watching channel 11, then switches to channel 34 by typing “3” . . . “4” using the hand-held remote controller 38, and then subsequently presses the “switch to last channel” button on the hand-held remote controller 38, the television 32 returns to channel 11. In a multi-channel feed, using the “switch to last channel” operation ensures that the television 32 returns to the correct channel of the entertainment audio/video content datastream 26 after the viewing of the selected patient-specific content is complete. Alternatively, the current channel can be determined by suitable feedback (not shown) to the controller 42, and then the controller can operate the transmitter 58 to send remote control signals mimicking the hand-held remote controllers operation selecting that channel to switch back.

With continuing reference to FIG. 5 and with further reference to FIG. 6, the controller 42 can select when to interrupt the entertainment audio/video datastream 26 in various ways. In the example of FIG. 6, the controller 42 elects to interrupt the entertainment datastream 26 responsive to receipt of urgent patient-specific content 70. The term “urgent” means here that the content is sufficiently important that the controller 42 elects to interrupt the entertainment datastream 26 to display it. For example, the urgent patient-specific content 70 may a scheduled goal module on the care plan schedule 52, or may be a message telling the patient 10 to breath into a bag responsive to hyperventilation detected by the sensors 14, or so forth. A portion 72 of the entertainment content 26 indicated by a dashed box is replaced by the urgent patient-specific content 70. Typically, the replaced entertainment content portion 72 is lost—the patient 10 never sees it because it is replaced by the urgent patient-specific content 70. In some embodiments, however, the entertainment device includes the digital video recorder (DVR) 34 or other recording device that provides time-shifting capability. As typical DVR devices are also remotely controllable, the controller 42 can readily implement time-shifting by having the transmitter 58 mimic suitable remote control signals to cause the DVR 34 to cache the entertainment content 26 while the urgent patient-specific content 70 is presented, and then mimic suitable remote control signals to cause the DVR 34 to initiate play back starting at the point where the entertainment content datastream 26 was interrupted.

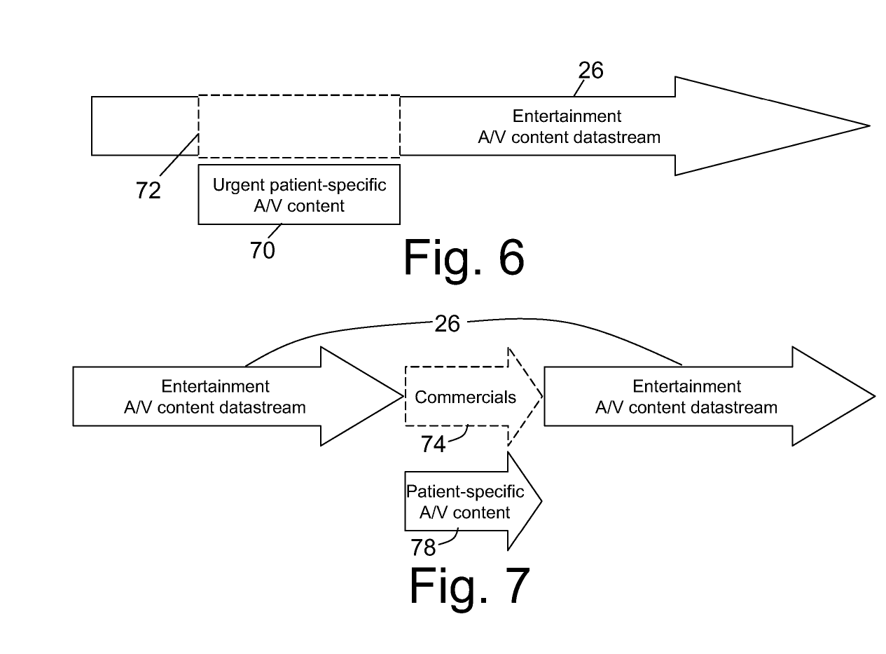

With continuing reference to FIGS. 1 and 5 and with further reference to FIG. 7, in another example the controller 42 elects to substitute patient-specific content for commercials 74 optionally included in the entertainment audio/video datastream 26. Commercials 74 can be expected, for example, if the entertainment audio/video datastream 26 is a broadcast television signal, cable television signal, or other television signal whose availability is supported by inclusion of advertising content. This approach makes use of an optional commercial detector 76 (FIG. 1) to detect when commercials 74 begin and end in the datastream 26. Some commercial detectors contemplated for use as the commercial detector 76 are disclosed in DeLuca, U.S. Pat. No. 5,973,723, Vogel, U.S. Publ. Appl. No. 2003/0145320 A1, and Trajkovic et al., U.S. Publ. Appl. No. 2002/0178444 A1, all three of which are incorporated herein by reference in their entirety. During commercials 74 detected by the commercials detector 76, selected patient-specific content 78 is substituted for the commercials 74, under the assumption that the content 78 is more valuable to the patient 10 than the commercials 74.

In some embodiments, the time-domain multiplexing is achieved by having the multiplexor 56 load the patient-specific content in a designated channel, and having the controller 42 switch to the designated channel during time intervals in which the patient-specific content is to be presented. In other embodiments, the multiplexor 56 inserts the time-domain multiplexed patient-specific content by directly overwriting the entertainment content in the entertainment content datastream 26. For example, with reference again to FIG. 7, rather than switching to a designated patient-specific content channel during commercials, the time-domain multiplexor 56 can instead overwrite the commercials 74 in the currently presented entertainment channel with the patient-specific content 78, which is then presented by continuing to present the currently presented entertainment channel without a change of channel. It is contemplated to buffer the entertainment audio/video content datastream 26 to provide time for detecting the commercials and making the substitution of patient-specific content for the commercials. Another approach for time-domain multiplexing is for the multiplexor 56 to act as an audio/video switch that receives two or more audio/video signals (such as the entertainment A/V datastream 26 and an additional datastream containing patient-specific content) and conveys a selected one of the received signals to the television 32 for presentation to the patient. The signal selection can be made through a suitable wired or wireless signal provided by the patient-specific content controller 42.

In another contemplated embodiment, the message is flashed on the screen for a very short time duration to provide subliminal motivations. If the patient-specific content is of a type that should be acknowledged by the patient, then the content may be displayed until such feedback is received. For example, a message to take a pill dispensed by the automatic pill dispenser 53 may scroll across the television screen until a sensor on the automatic pill dispenser 53 detects that the patient has removed the dispensed pill from the automatic pill dispenser 53. The sensor may be, for example, a pressure or contact sensor disposed on the bottom of a dispensing tray that detects when the weight of, or contact with, the dispensed pill is removed by the patient taking the pill off the dispensing tray. In some such embodiments, the presentation of the patient-specific content may change to become more intrusive if the patient does not respond immediately. For example, the message “Please take your pill” may start out as a scrolling steady message, and then switch to a blinking or flashing message if no patient response is detected, and if still no response is detected an audible signal may be activated. If the patient response is too long delayed, it is also contemplated to transmit a signal to a nurses' station (in a hospital setting) or to a telephonic Lifeline (in the case of a patient at home), or to another third party.

If the patient-specific content is urgent (for example, a warning that the patient needs to stop and rest to control a palpitating heartbeat detected by cardiac sensors and the data analyzer 44) then suitable audio or visual cues are optionally provided to ensure that the receives the content promptly. For example, the television may make several loud “beeps” to get the patient's attention. If the patient-specific content is urgent and the patient is outdoors or otherwise away from the television 32, it is contemplated to use an alternate communication pathway such as a cellular telephone to deliver the urgent patient-specific content.

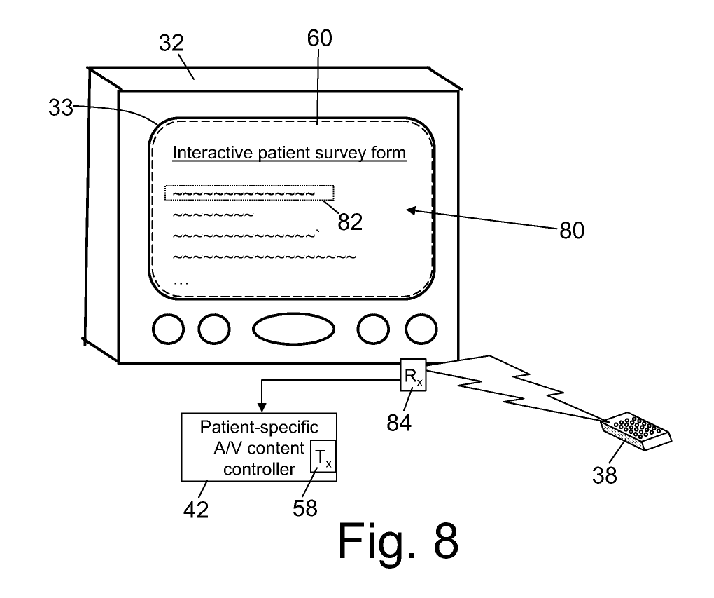

With reference to FIG. 1 and with further reference to FIG. 8, it is contemplated for the patient-specific content presented with the entertainment content datastream 26 may be interactive content. In FIG. 8, the time-domain multiplexing approach of FIG. 5 is used. FIG. 8 shows patient-specific content in the form of an interactive patient survey form 80. The form suitably displays highlighting 82 to indicate a question currently under consideration. In one approach, the question is formatted to be answerable by selecting a number. For example, a question relating to difficulty in breathing could be suitably formulated as follows:

Question 1: Are you experiencing difficulty in breathing?
Press “1” to indicate no difficulty;
Press “2” to indicate occasional difficulty;
Press “3” to indicate frequent difficulty;
Press “4” to indicate continuous difficulty.
   The patient 10 then presses the appropriately responsive number button on the hand-held remote controller 38. A receiver 84 operatively coupled with the controller 42 and located close to the television 32 detects the wireless signal generated by the patient's pushing the appropriate button, and the controller 42 receives this answer.

The illustrated embodiments are in the context of the hospital room 12 or other clinical setting. However, it is contemplated to use such approaches for integrating patient-specific content into entertainment audio/video content datastreams in other settings, such as in the home of a person receiving care. Moreover, the illustrated and described approaches for inserting patient-specific content into the entertainment audio/video content datastream are illustrative examples, and other approaches can be used. Yet further, the illustrated and described sources of patient-specific content for insertion are illustrative examples, and other sources and types of patient-specific content can be used. The patient-specific content may also be delivered to other parties besides the patient, such as to the patient's doctor or to the patient's family members (preferably conditional upon patient consent to release of such information or other protections for the privacy of patient medical information).

An advantage of presenting the patient-specific content with the entertainment audio/video content datastream 26 on the entertainment device 30 is that the patient 10 typically engages in this activity a substantial amount of time (typically for a longer time, for example, than the cumulative time of nurse and doctor visits). Moreover, the patient 10 is typically paying attention to the entertainment content, and hence is likely to absorb the presented patient-specific content as well. However, the patient typically does not constantly use the entertainment device 30—rather, the patient may spend significant time in other activities such as sleeping, reading, or so forth. In some embodiments, the patient-specific content is only presented when the entertainment device 30 is already operating to present the entertainment audio/video content datastream 26. In these embodiments, when the device 30 is turned off (or is placed into a “sleep” mode, or is otherwise set to be inoperative for presenting the entertainment audio/video content datastream 26) it follows that no patient-specific content is presented.

In other embodiments, the controller 42 may be configured to operate the transmitter 58 to send suitable mimicked remote control signals to turn on the entertainment device 30, or a selected portion thereof such as the television 32. The controller 40 may, for example, selectively turn on the television 32 when the controller 42 receives urgent patient-specific content, such as advice for the patient on how to deal with a current asthma attack or other acute problem. It is also contemplated for the entertainment device 30 to be left on constantly, and for it to display only patient-specific content (optionally with no audio portion, optionally with the display 33 dimmed, or otherwise configured to be relatively unobtrusive) when the patient “turns off” the presentation of the entertainment audio/video content datastream 26.

Still further, it is contemplated for the controller 42 and/or multiplexor 56 to actively intervene with the viewing by the patient 10 of the entertainment audio/video content datastream 26. For example, the controller 42 may be configured to limit the patient's viewing during nighttime hours (e.g., after 10:00 p.m. and before 7:00 a.m.). In some embodiments, the controller 42 does so by operating the transmitter 58 to send suitable mimicked remote control signals to turn off the entertainment device 30, or a selected portion thereof such as the television 32, or to put the device to sleep or otherwise make it inoperative. In some embodiments, the controller 42 used in conjunction with a heart patient or other patient with a sensitive condition is configured to monitor the patient's vital signs via the sensors 14, and to cause the entertainment device 30 to switch from a channel known for showing stimulating content (such as sporting events, X-rated movies, or the like) to a channel known for showing more soothing content if the monitored vital signs show that the patient is getting overly stimulated. Instead of switching to a soothing channel of the entertainment audio/video content datastream 26, the controller 42 may switch to soothing patient-specific content, for example using the time-domain multiplexing approach diagrammatically depicted in FIG. 5.

In the illustrated examples, the controller 42 operates the entertainment device 30 via the wireless transmitter 58 mimicking signals of the wireless handheld remote controller 38. In other contemplated embodiments, more direct operational control is provided, for example by including suitable wired control connections between the controller 42 and the entertainment device 30, or by integrating the controller 42 into the entertainment device 30. Similarly, the multiplexor 56 can be integrated into the entertainment device 30, or into the controller 42. Other physical embodiments can be used. For example, in some embodiments a computer or computer network embodies the data analyzer 44, storage 40, and message entry interface 54. Such a computer or computer network can also optionally store the care plan schedule 52 and care plan modules 50, and can optionally embody the controller 42, and can optionally embody the multiplexor 56. The storage and data components 40, 50, 52 are suitably embodied by memory or storage elements of the computer, such as RAM, hard disk space, or so forth. The processing components 42, 44 are suitably embodied by one or more general purpose microprocessors of a computer or computer network executing suitable software. The message entry interface 54 is suitably embodied by interface devices of a computer such as a keyboard, mouse, and so forth, or by a dumb network terminal, operating in conjunction with suitable executing software. The multiplexor 56 is suitably embodied by one or more general purpose microprocessors of a computer or computer network executing suitable software in embodiments in which the entertainment datastream 26 is digital, or may be embodied by an add-on application-specific integrated circuitry (ASIC) or other added or connected hardware for processing a digital datastream faster or for processing an analog datastream. In some embodiments, it is contemplated for the care plan audio/video modules 50 to be integrated closely with the entertainment device 30, for example stored as content on the hard drive or other non-volatile storage of the DVR 34.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

Having thus described the preferred embodiments, the invention is now claimed to be:

1. A medical information delivery system configured for operation in conjunction with an entertainment device (30) configured to present an entertainment audio/video content datastream (26), the medical information delivery system comprising:
a means (42, 56, 58, 76) for inserting additional content into the entertainment audio/video content datastream; and
a means (42, 44, 52, 54) for selecting patient-specific content for insertion into the entertainment audio/video content datastream.

2. The medical information delivery system as set forth in claim 1, wherein the inserting means (42, 56, 58, 76) includes a multiplexor (56), and the selecting means (42, 44, 52, 54) includes a selection controller (42).

3. The medical information delivery system as set forth in claim 2, wherein the means (42, 44, 52, 54) for selecting patient-specific content prioritizes patient-specific content and selects patient-specific content for insertion based on content priority.

4. The medical information delivery system as set forth in claim 2, wherein the controller (42) includes:
a wireless transmitter (58) mimicking wireless outputs of a hand-held remote controller (38) configured to control the entertainment device (30).

5. The medical information delivery system as set forth in claim 4, wherein the multiplexor (56) is configured to insert the additional content into a designated channel of the entertainment audio/video content datastream (26), and the controller (42) is configured to mimic wireless outputs that cause the entertainment device (30) to display the designated channel.

6. The medical information delivery system as set forth in claim 5, wherein the controller (42) is configured to mimic wireless outputs that cause the entertainment device (30) to display the designated channel in one of a sub-window (62) and a main window (60).

7. The medical information delivery system as set forth in claim 2, wherein the multiplexor (56) is configured to insert the additional content as text superimposed on presented video of the entertainment audio/video content datastream (26).

8. The medical information delivery system as set forth in claim 2, wherein the multiplexor (56) is configured to substitute pixels corresponding to additional content for selected video pixels of the entertainment audio/video content datastream (26).

9. The medical information delivery system as set forth in claim 2, wherein the multiplexor (56) is configured to insert the additional content into the entertainment audio/video content datastream (26) as one or more of (i) video displayed in a sub-window, (ii) a scrolling banner, (iii) superimposed text, and (iv) substitute streaming video.

10. The medical information delivery system as set forth in claim 2, further including:
a data analyzer (44) that analyzes one or more patient measurements to cause the controller (42) to select patient-specific medical advice content in accordance with the analyzed measurements.

11. The medical information delivery system as set forth in claim 10, further including:
a wireless body sensor network comprising wirelessly intercommunicating sensors (14) configured to generate at least some of the one or more patient measurements.

12. The medical information delivery system as set forth in claim 2, further including:
a care module (50) comprising pre-recorded medically related content that is selectable by the controller (42); and
a care plan schedule (52), the controller selecting the care module as the patient-specific content for insertion into the entertainment audio/video content datastream (26) based on the care plan schedule.

13. The medical information delivery system as set forth in claim 2, further including:
a care module (26) comprising interactive medically related content (80) that is selectable by the controller (42), the controller further including a wireless receiver (84) for receiving wireless responses transmitted by a hand-held remote controller (38).

14. The medical information delivery system as set forth in claim 2, further including:
storage (40) for storing patient-specific content that is selectable by the controller (42); and
a user interface (54) for inputting patient-specific content to the storage (40).

15. The medical information delivery system as set forth in claim 1, further including:
a commercial detector (76) for detecting commercials in the entertainment audio/video content datastream (26), the patient-specific content replacing the commercials in the presentation of the entertainment audio/video content datastream.

16. A medical information delivery method comprising:
presenting entertainment audio/video content (26) via an entertainment device (30); and
selectively presenting patient-specific content via the entertainment device with the presenting of the entertainment audio/video content.

17. The medical information delivery method as set forth in claim 16, wherein the selective presenting of the patient-specific content includes:
automatically modifying a display (60) of the entertainment audio/video content (26) to allow for the presentation of the patient-specific content.

18. The medical information delivery method as set forth in claim 16, wherein the selective presenting of the patient-specific content includes at least one of:
displaying textual patient-specific content (64) superimposed on presented video of the entertainment audio/video content datastream (26);
displaying patient-specific content as a scrolling banner (66) superimposed on presented video of the entertainment audio/video content datastream (26);
displaying patient-specific content in a sub-window (62) superimposed on a main window (60) presenting video of the entertainment audio/video content datastream (26); and
substituting patient-specific video content (70, 78) for selected video portions (72, 74) of the entertainment audio/video content datastream (26).

19. The medical information delivery method as set forth in claim 16, further including:
acquiring one or more patient measurements; and
selecting the patient-specific content for presentation in accordance with the one or more patient measurements.

20. The medical information delivery method as set forth in claim 16, further including:
receiving patient responses to the presented patient-specific content via a hand-held remote controller (38) configured to control the entertainment device (30).

21. The medical information delivery method as set forth in claim 16, further including:
scheduling a presentation time for a care module (50) comprising pre-recorded medically related content, the presenting of patient-specific content including presenting the care module at the scheduled presentation time.

22. The medical information delivery method as set forth in claim 16, wherein the selective presenting of patient-specific content via the entertainment device is timed to coincide with commercial advertisement segments of the presented entertainment audio/video content (26).

23. The medical information delivery method as set forth in claim 16, wherein the presenting of entertainment audio/video content (26) via an entertainment device (30) comprises presenting continuously streamed broadcast audio/video, and the selective presenting of patient-specific content via the entertainment device with the presenting of the entertainment audio/video content comprises substituting patient-specific content (70, 78) for selected time intervals (72, 74) of the continuously streamed broadcast audio/video content.

24. The medical information delivery method as set forth in claim 16, wherein the presenting of entertainment audio/video content (26) via an entertainment device (30) comprises presenting continuously streamed broadcast audio/video, and the selective presenting of patient-specific content via the entertainment device with the presenting of the entertainment audio/video content comprises spatially overlaying patient-specific content (62, 64, 66) onto the video component (60) of the continuously streamed broadcast audio/video content.

25. An information delivery system comprising:
an entertainment device (30) comprising one or more components (32, 34) configured to present an entertainment audio/video content datastream (26); and
one or more additional components (40, 42, 44, 50, 52, 56) configured to select and insert viewer-specific content unrelated to the entertainment content into the entertainment audio/video content datastream for presentation by the entertainment device along with the entertainment audio/video content datastream (26).

26. The information delivery system as set forth in claim 25, wherein the one or more additional components (40, 42, 44, 50, 52, 56) are configured to select and superimpose viewer-specific content on presented video of the entertainment audio/video content datastream (26) shown on a screen or display (33) of the entertainment device (30).

27. The information delivery system as set forth in claim 25, wherein the one or more additional components (40, 42, 44, 50, 52, 56) are configured to select and time-domain multiplex presentation of the viewer-specific content and presentation of the entertainment audio/video content datastream (26) via the entertainment device (30).

28. The information delivery system as set forth in claim 25, further including one or more monitors (14) which monitor a viewer's physiological condition, the viewer-specific content being selected in accordance with the monitored physiological condition.

29. The information delivery system as set forth in claim 25, wherein the one or more additional components (40, 42, 44, 50, 52, 56) include:
one or more components (14, 52, 54) configured to acquire viewer-specific information, the viewer-specific content being constructed based on the acquired viewer-specific information.
